(19) United States
(12) Patent Application Publication
(10) Pub. No.: US 2017/0310886 A1
(43) Pub. Date: Oct. 26, 2017

(54) METHOD AND SYSTEM FOR PERFORMING A VIRTUAL CONSULTATION

(71) Applicant: J.A.K. Investments Group LLC, Cincinnati, OH (US)

(72) Inventor: Darren Hurst, Cincinnati, OH (US)

(21) Appl. No.: 15/448,033

(22) Filed: Mar. 2, 2017

Related U.S. Application Data
(60) Provisional application No. 62/327,507, filed on Apr. 26, 2016.

(51) Int. Cl. H04N 5/232; A61B 5/00; H04N 7/18; G06T 7/00; H04L 29/08

(52) U.S. Cl. CPC H04N 5/23222; A61B 5/0077; A61B 5/7246; H04N 5/23293; G06T 7/0014; H04N 7/183; A61B 5/0013; A61B 2576/00; G06T 2207/10004; G06T 2207/30196; G06T 2210/41; H04L 67/06; H04L 67/10; G06T 2207/30101

(57) ABSTRACT

A software application which when executed by a mobile device processor, causes the mobile device processor to present an outline of a part of a human body on a display of the mobile device; capture an image of the part of the human body with a camera of the mobile device; determine whether the captured image is acceptable based on at least one of contrast, light, warping, angle and degree of fit with the outline; and transfer the captured image over a network to a server in communication with a reader station if the captured image is found acceptable.

### BACKGROUND

Many people who have symptomatic or asymptomatic varicose veins currently have to obtain and schedule a face-to-face consultation with a physician for initial evaluation of varicose veins. The process of scheduling and the time commitment of the clinical visit often serves as a significant barrier to obtaining appropriate treatment. Many people are unaware of their options for therapy, and because varicose veins are not life threatening, they choose to forgo investigation of treatment of their problem and revert to benign neglect of their issue until it becomes severe and more difficult to treat. This problem regarding the barrier to obtaining appropriate treatment is prevalent in other medical fields as well.

### SUMMARY

In view of the foregoing, a method and system to allow for the performance of a virtual consultation to determine the appropriateness of pursuit of further in depth face to face consultation, for example, for the management of varicose vein disease of the lower extremities is provided. The method and system, however, can be useful in other medical practices as well, such as dermatology, ophthalmology, dentistry and others. The system can include a software application capable of being run on a mobile device. The software application is configured to provide the operator of the mobile device a prompt to indicate the location on the patient's body of which an image is to be captured. The software application would then present an outline of the appropriate location on a display of the mobile device. The software application would then instruct the operator to capture an image. The software application would then evaluate the captured image, which was captured by the operator using the mobile device, for (1) contrast, (2) light, (3) warping, (4) angle and/or (5) degree of fit with the outline that was presented on the display. After evaluating the captured image, the software application would then determine whether the captured image was acceptable. If the captured image was not acceptable, e.g., there is not enough contrast in the image, then the software application returns to provide the operator a prompt to capture another image of the extremity. If the software application finds the image to be acceptable, then the image is saved and the software application may instruct the operator to capture an image of another location on the patient. The software application continues to operate in the manner described above until acceptable images are captured for each area of interest and then these images are submitted over a network to a reading station for view by a clinician.

The software application allows the patient or his significant other to submit clinical quality pictures of his vein problem via a mobile device through HIPPA compliant application software.

### BRIEF DESCRIPTION OF THE DRAWINGS

FIG. 1 schematically depicts a system for providing a virtual consultation.

FIG. 2 is a view of a mobile device where a display of the mobile device presents general patient information requests.

FIG. 3 is a view of the mobile device where the display of the mobile device presents clinical information requests/questions.

FIG. 4 is a view of the mobile device where the display of the mobile device presents instructions to an operator of the device.

FIG. 5 is a flow diagram depicting a method for acquiring images for performing a virtual consultation.

FIGS. 6 and 7 are views of the mobile device where the display of the mobile device presents instructions to a photographer.

FIGS. 8 and 9 are views of the mobile device when the photographer is in the process of taking an image of a patient.

### DETAILED DESCRIPTION

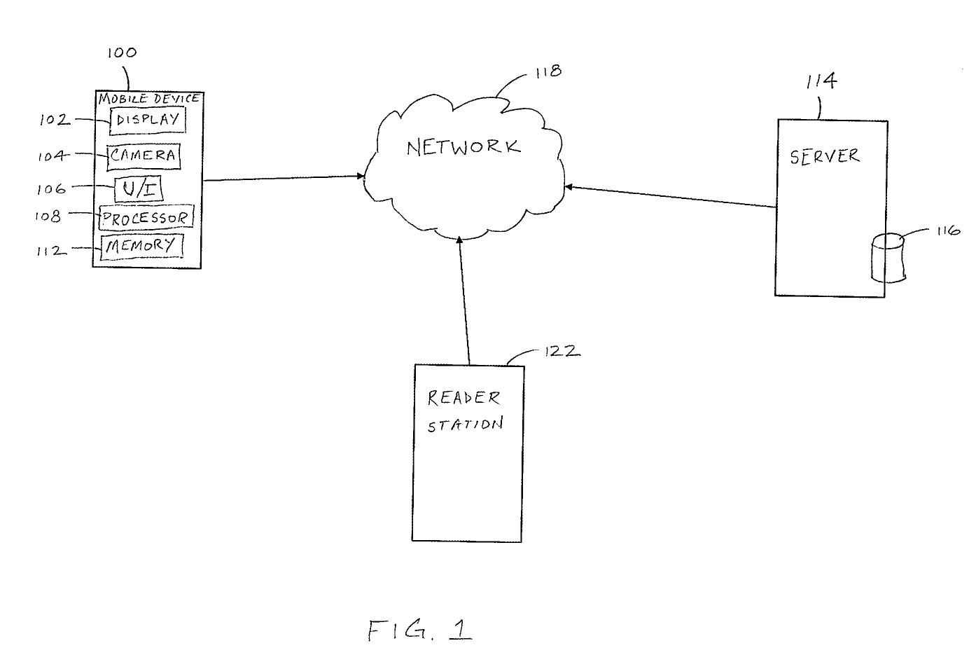

FIG. 1 depicts a general environment in which a mobile device 100, such as a smart phone, tablet, etc., which includes a display 102, a camera 104, a user interface 106, processor 108, and a memory 112, communicates with a server 114, which communicates with a database 116, through a network 118, such as the internet. A software application, which can be downloaded from the network 118, runs on the processor 108 of the mobile device 100 and allows an operator of the mobile device 100 to submit clinical quality images via the mobile device 100 and the network 118 to HIPPA compliant application software on the server 114. Physicians can access the images through the network 118 or directly from the server 114 and view the images on a reader station 122.

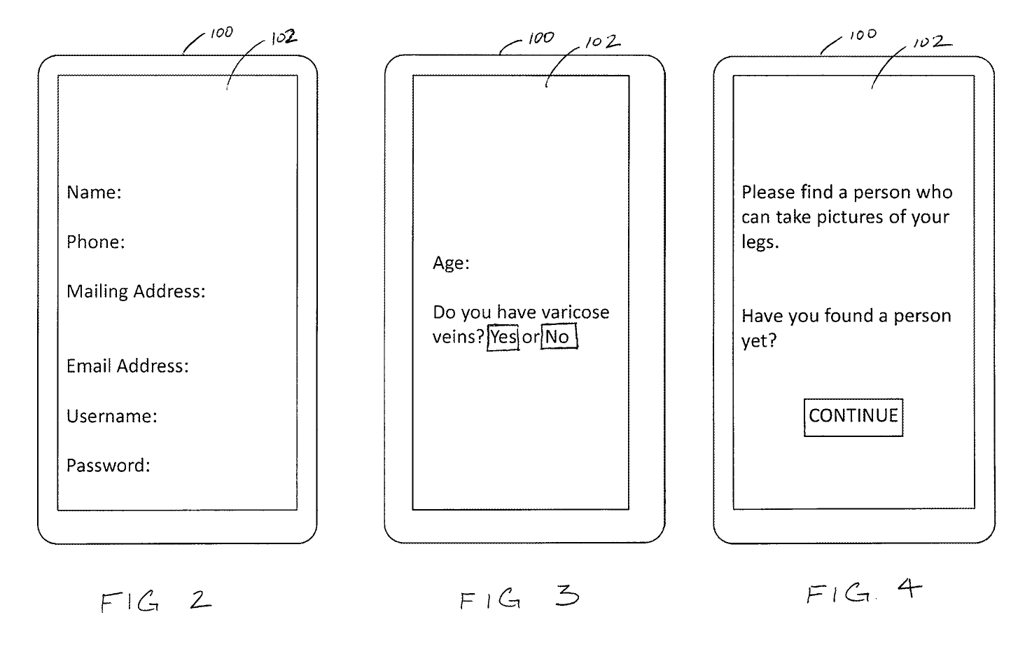

The software application running on the processor 108 of the mobile device 100 is configured to direct an operator of the mobile device 100 to obtain images of a human, hereinafter “patient,” in a fashion that would allow a clinician to review the images and provide a “virtual consult.” For example, the software application can instruct the operator of the mobile device 100 to obtain images of the legs of the patient to provide a virtual consult for the patient's varicose vein disease. Other locations on the body, however, can be captured that would be relevant to other medical fields. With reference to FIG. 2, the software application is configured to present requests for general information from the patient on the display 102 of the mobile device 100. These general patient information requests can include: name, phone number, mailing address, email address, username and password. The software application records responses to the general patient information requests, which can be input by the patient via the user interface 106, and stores the responses on the memory 112.

With reference to FIG. 3, the software application is further configured to present requests/questions for clinical information on the display 102 of the mobile device 100. These requests/questions can be directed to clinical information and can include:

(1) Age

(2) Do you have varicose veins? Yes or No

(3) Where are your varicose veins?
(a) Right leg
(b) Left leg
(c) Both legs

(4) Do you have any of the following symptoms:
(a) Pain
(b) Heaviness
(c) Itching
(d) Skin discoloration
(e) Swelling
(f) Restless legs
(g) Ulcers

(6) Have you ever had a surgery or procedure for your varicose veins?
(a) Surgery/stripping
   (i) Right leg
   (ii) Left leg
   (iii) Both legs
(b) Sclerotherapy
   (i) Right leg
   (ii) Left leg
   (iii) Both legs
(c) Ablation
   (i) Right leg
   (ii) Left leg
   (iii) Both legs
(d) Other

FIG. 3 depicts the mobile device 100 where the display 102 shows a screen with only the first two clinical information requests. The software application can be configured to present each clinical information request/question on the display 102 of the mobile device 100 as separate screens. The software application records responses to the clinical requests/questions, which can be input by the patient via the user interface 106, and stores the responses on the memory 112. The software application can also be configured to end the process if the patient answers “no” to question 2—“Do you have varicose veins?” Moreover, the content of the clinical information request/questions can be based on a particular medical field. The clinical information request/questions provided above are relevant to varicose veins. The clinical information request/questions may request information about the patient's eyes in an ophthalmology application, the patient's skin in a dermatology application, and the patient's teeth in a dentistry application, for example.

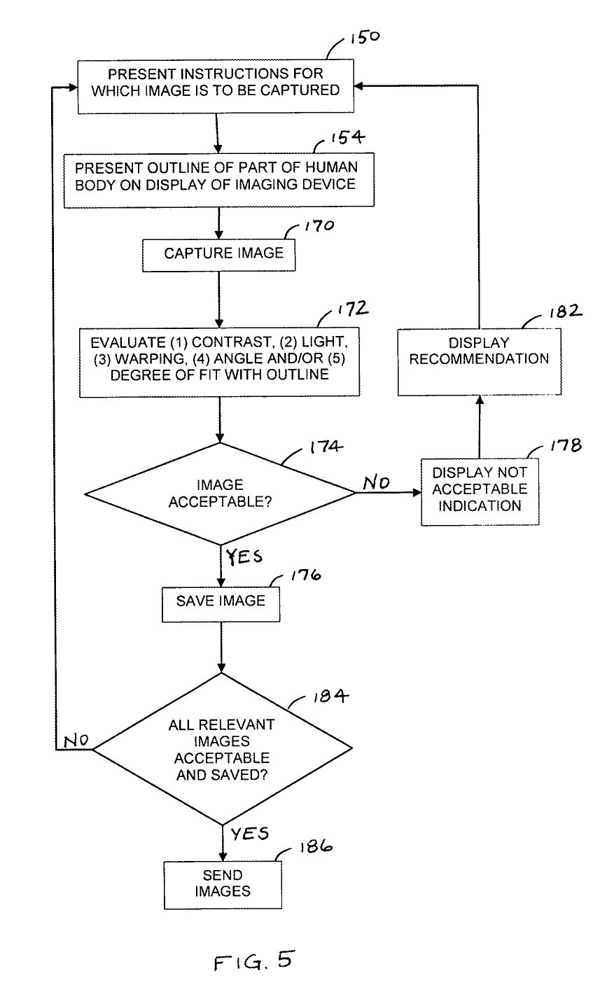

With reference to FIG. 4, after the responses to the clinical requests/questions have been recorded, the software application then presents a prompt for the patient to find another person, the photographer, to take images of the varicose veins. In some instances the photographer and the patient may be the same person. The software application is configured to assist the photographer to capture the appropriate images based on the responses to the clinical information request/questions. With regard to the example of a varicose veins virtual consult, the software application is configured to assist the photographer to capture the appropriate images based on the responses to the clinical information request/question (3) above, i.e., “Where are your varicose veins?” The software application is further configured to assist the photographer to align the mobile device 100, and more particularly the camera 104 on the mobile device, using outlines (discussed in more detail below), and to evaluate each image prior to sending the image to the server 114 over the network 118.

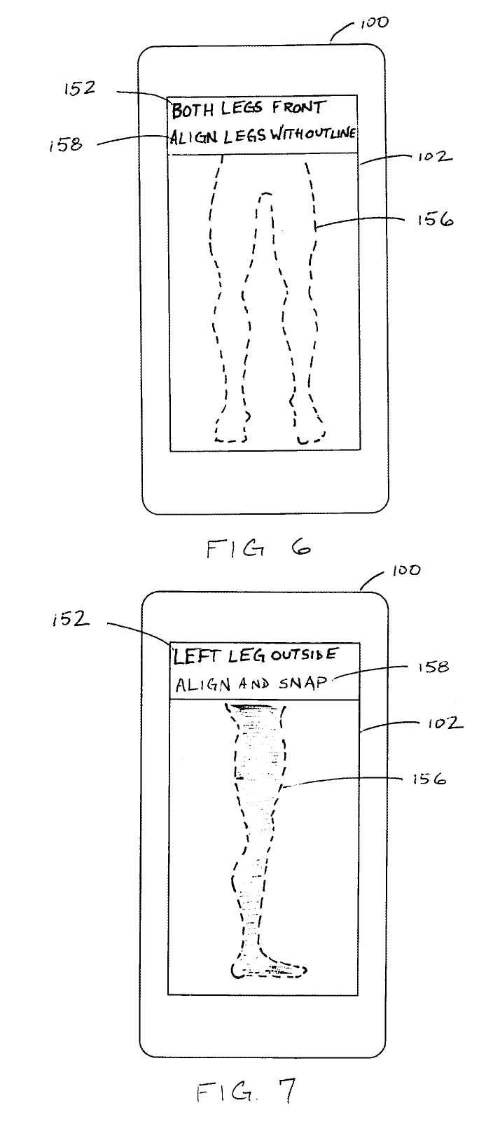

FIG. 5 depicts a flow diagram of a method for acquiring images for performing a virtual consultation. With reference to FIG. 5, at 150, the software application directs the processor 108 on the mobile device 100 to present instructions 152 (FIG. 6) on the display 102 of the mobile device 100 of which human extremity is to be imaged using the camera 104. FIG. 6 depicts the instructions 152 as “BOTH LEGS FRONT” to indicate that an image of the front of both legs of the patient is to be taken. The list of possible instructions include the following:

(1) BOTH LEGS FRONT

(2) BOTH LEGS REAR

(3) LEFT LEG OUTSIDE (see FIG. 7)

(4) LEFT LEG INSIDE

(5) RIGHT LEG OUTSIDE

(6) RIGHT LEG INSIDE

(7) CLOSE UP

With reference back to FIG. 5, at 154, the software application directs the processor 108 on the mobile device 100 to present an outline 156 of a human extremity on the display 102 of the mobile device 100. The outline 156 corresponds to the instructions 152 presented on the display 102. The outline 156 can be a dotted line (as shown in FIG. 6) or a shaded image (as shown in FIG. 7) of a typical human leg (in the instance of FIG. 7) or legs (in the instance of FIG. 6). Even though only outlines 156 for BOTH LEGS FRONT and LEFT LEG OUTSIDE are depicted, one of ordinary skill in the art would understand that other outlines could be presented on the display 100 with the appropriate instructions. For example, instructions such as “BOTH LEGS REAR” could be presented on the display 102 and an outline similar to the outlines shown in FIG. 6 or 7 but depicting the rear of both legs would be presented on the display 102.

FIGS. 6 and 7 depict the instructions 152, e.g., “BOTH LEGS FRONT” in FIG. 6 and “LEFT LEG OUTSIDE” in FIG. 7, being displayed simultaneously with the appropriate outlines 156. However, the instructions could be displayed on a prior screen, such as “TAKE A PICTURE OF THE FRONT OF BOTH LEGS,” and the photographer could acknowledge thee instructions through the user interface 106, for example a “NEXT” button could be displayed. The software application would then change the presentation on the display 102 to only show the outline 156 of both front legs.

The instructions 152 and the outlines 156 presented on the display 100 can be based on the recorded responses to the clinical requests/questions discussed above. For example, if the patient responds to the clinical information request/question (3) above, i.e., “Where are your varicose veins?” with “Right leg,” then the software application directs the processor 108 to provide instructions 152 and to display corresponding outlines 156 of the following: BOTH LEGS FRONT, BOTH LEGS REAR, RIGHT LEG OUTSIDE, RIGHT LEG INSIDE and CLOSE UP, which can be optional. Likewise, if the patient responds to the clinical information request/question (3) above, i.e., “Where are your varicose veins?” with “Left leg,” then the software application directs the processor to provide instructions 152 and to display corresponding outlines 156 of the following: BOTH LEGS FRONT, BOTH LEGS REAR, LEFT LEG OUTSIDE, LEFT LEG INSIDE and CLOSE UP, which can be optional. Moreover, if the patient responds to the clinical information request/question (3) above, i.e., “Where are your varicose veins?” with “Both legs,” then the software application directs the processor 108 to provide instructions 152 and to display corresponding outlines 156 of the following: BOTH LEGS FRONT, BOTH LEGS REAR, LEFT LEG OUTSIDE, LEFT LEG INSIDE, RIGHT LEG OUTSIDE, RIGHT LEG INSIDE and CLOSE UP, which can be optional.

The software application assists the photographer in alignment of the camera 104 on the mobile device 100 by presenting the appropriate outline 156 on the display 102. The software application can also direct the processor 108 on the mobile device 100 to present alignment instructions 158 such as “ALIGN LEGS WITH OUTLINE” depicted in FIG. 6.

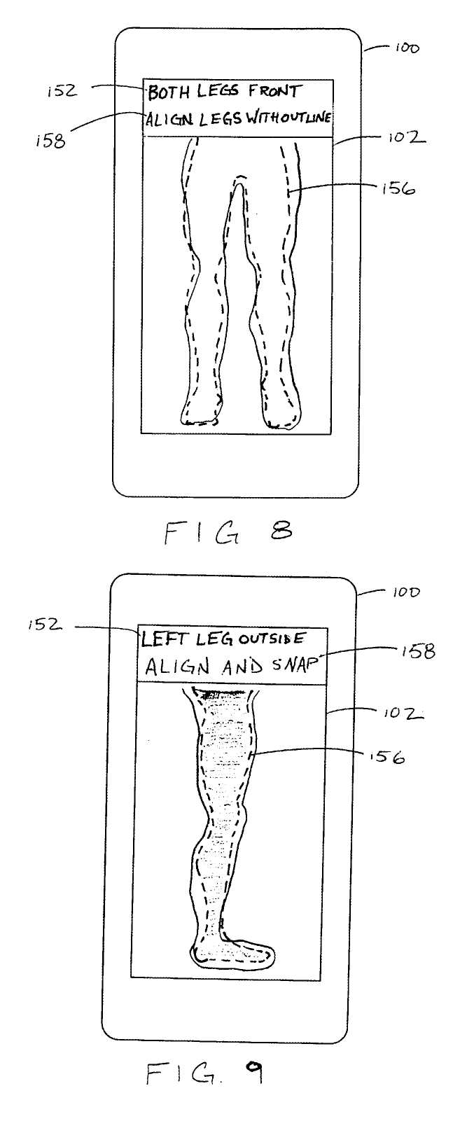

With reference back to FIG. 5, at 170, an image of the patient's extremity is then captured with the mobile device 100. The software application can be configured to operate the camera 104 to allow the photographer to take a picture in a manner similar to the camera application already residing on the mobile device. The software application can also direct the processor 108 on the mobile device 100 to present alignment instructions and image capturing instructions 158 such as “ALIGN AND SNAP” depicted in FIG. 7. FIGS. 8 and 9 depict views of the mobile device 100 when the photographer is in the process of taking an image of the patient. The patient's leg(s) in FIGS. 8 and 9 are shown in solid lines. As mentioned above, the software application can be modified to present different clinical information request/questions. The software application can also be modified to present different outlines 156 on the display 102 of the mobile device. For example, the outline could be an outline of a human eye (or part thereof) in an ophthalmology application, the outline could be an outline of a human face in a dermatology application, and the outline could be of human teeth in a dentistry application.

After the image is captured, the software application, at 172, then evaluates the captured image. This evaluation is useful to determine if the captured image is acceptable for a clinician to review. The software application can be configured to evaluate the captured image for the following factors: (1) contrast, (2) light, (3) warping, (4) angle and (5) degree of fit with the outline. The software application, at 174, then determines whether the captured image is acceptable, which can be based on one or more of the aforementioned factors. These factors can be weighted to provide a score and if this score is above a predetermined threshold, then the captured image is determined to be acceptable and is saved, at 176, in the memory 112 of the mobile device 100. If the captured image is not found to be acceptable, then the software application can provide an indication that the image was not acceptable, at 178, and then eventually revert to step 150. At 182, prior to reverting back to step 150, the software application can direct the processor 108 to provide recommendations to the photographer based on the evaluation performed at step 172. For example, if the contrast in the captured image did not meet a predetermined threshold the processor 108 can present instructions on the display 102 to increase the contrast based on the camera application already running on the mobile device. If the lighting in the captured image did not meet a predetermined threshold, for example, the processor 108 can present instructions on the display 102 to move to an area with better lighting.

The software application, at 184, determines whether a number of relevant captured images have been found acceptable (per step 174) and have been saved. This determination can be based on the patient's response to the clinical information request/questions, for example clinical information request/question (3) above, i.e., “Where are your varicose veins?” For example, if the patient responds to the clinical information request/question (3) above, i.e., “Where are your varicose veins?” with “Right leg,” then the software application directs the processor 108 to determine whether acceptable images have been captured of (1) the front of both legs, (2) the rear of both legs, (3) the outside of the right leg, and (4) the inside of the right leg, for example. If further images are needed to be saved, then the software application reverts the process back to step 150. If no further images are needed, then, at 186, the software application instructs the processor 108 to send the acceptable captured images over the network 118 to the server 114 for retrieval by the reader station 122.

By using the aforementioned software application running on the processor 108 of the mobile device 100, the patient can provide the clinician the appropriate images to perform a virtual consultation so that the clinician can determine whether a face-to-face consultation is appropriate.

It will be appreciated that variations of the above-disclosed and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A computer-implemented method for acquiring images for performing a virtual consultation, the method comprising:
presenting an outline of a part of a human body on a display of a mobile device having a camera and running a software application;
capturing an image of the part of the human body with the camera;
using the software application, determining whether the captured image is acceptable based on at least one of contrast, light, warping, angle and degree of fit with the outline; and
if the captured image is found acceptable, transferring the captured image over a network to a server in communication with a reader station.

2. The method of claim 1, wherein the mobile device is a mobile phone.

3. The method of claim 1, further comprising presenting instructions of the part of the human body to be imaged on the display of the mobile device.

4. The method of claim 3, wherein the instructions and the outline are presented simultaneously on the display of the mobile device.

5. The method of claim 1, wherein determining whether the captured image is acceptable is based on each of contrast, light, warping, angle and degree of fit with the outline.

6. The method of claim 1, further comprising:
if the captured image is not found acceptable, then presenting a recommendation on the display of the device.

7. The method of claim 1, further comprising
presenting questions to be answered by the person on the display of the mobile device;
recording answers to the questions; and
presenting instructions of the part of the human body to be imaged on the display of the mobile device based on the recorded answers.

8. The method of claim 7, further comprising:
using the software application, determining whether a number of relevant captured images have been found acceptable, wherein the number of relevant captured images is based on the recorded answers.

9. A non-transitory storage medium storing instructions which when executed by a mobile device processor, causes the mobile device processor to:
present an outline of a part of a human body on a display of the mobile device;
capture an image of the part of the human body with a camera of the mobile device;
determine whether the captured image is acceptable based on at least one of contrast, light, warping, angle and degree of fit with the outline; and
transfer the captured image over a network to a server in communication with a reader station if the captured image is found acceptable.

10. The non-transitory storage medium of claim 9 further causes the mobile device processor to present instructions of the part of the body to be imaged on the display of the mobile device.

11. The non-transitory storage medium of claim 10 further causes the mobile device processor to present the instructions and the outline simultaneously on the display of the mobile device.

12. The non-transitory storage medium of claim 9 further causes the mobile device processor to determine whether the captured image is acceptable based on each of contrast, light, warping, angle and degree of fit with the outline.

13. The non-transitory storage medium of claim 9 further causes the mobile device processor to present a recommendation on the display of the device if the captured image is not found acceptable.

14. The non-transitory storage medium of claim 9 further causes the mobile device processor to:
present questions to be answered by the person on the display of the mobile device;
record answers to the questions; and
present instructions of the part of the human body to be imaged on the display of the mobile device based on the recorded answers.

15. The non-transitory storage medium of claim 14 further causes the mobile device processor to determine whether a number of relevant captured images have been found acceptable, wherein the number of relevant captured images is based on the recorded answers.
